SEQUENCE LISTING (24 sequences)

SEQ ID NO: 1 - protein, 107 aa, synthetic construct [sequence omitted]

SEQ ID NO: 2 - protein, 118 aa, synthetic construct [sequence omitted]

SEQ ID NO: 3 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 4 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 5 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 6 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 7 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - DNA, 321 bp, synthetic construct
  source 1..321: mol_type = other DNA
gatattcagatgacccagagcccgagcagcctgagcgcgagcgtgggcgatcgcgtgaccattacctgccgcgcgagccaggatgtgagcaccgcggtggcgtggtatcagcagaaaccgggcaaagcgccgaaactgctgatttatagcgcgagctttctgtatagcggcgtgccgagccgctttagcggcagcggcagcggcaccgattttaccctgaccattagcagcctgcagccggaagattttgcgacctattattgccagcagtatctgtatcatccggcgacctttggccagggcaccaaagtggaaattaaa

SEQ ID NO: 9 - DNA, 354 bp, synthetic construct
  source 1..354: mol_type = other DNA
gaagtgcagctggtggaaagcggcggcggcctggtgcagccgggcggcagcctgcgcctgagctgcgcggcgagcggctttacctttagcgatagctggattcattgggtgcgccaggcgccgggcaaaggcctggaatgggtggcgtggattagcccgtatggcggcagcacctattatgcggatagcgtgaaaggccgctttaccattagcgcggataccagcaaaaacaccgcgtatctgcagatgaacagcctgcgcgcggaagataccgcggtgtattattgcgcgcgccgccattggccgggcggctttgattattggggccagggcaccctggtgaccgtgagcagc

SEQ ID NO: 10 - DNA, 324 bp, synthetic construct
  source 1..324: mol_type = other DNA
gaaattgtgctgacccagagcccgggcaccctgagcctgagcccgggcgaacgcgcgaccctgagctgccgcgcgagccagcgcgtgagcagcagctatctggcgtggtatcagcagaaaccgggccaggcgccgcgcctgctgatttatgatgcgagcagccgcgcgaccggcattccggatcgctttagcggcagcggcagcggcaccgattttaccctgaccattagccgcctggaaccggaagattttgcggtgtattattgccagcagtatggcagcctgccgtggacctttggccagggcaccaaagtggaaattaaa

SEQ ID NO: 11 - DNA, 363 bp, synthetic construct
  source 1..363: mol_type = other DNA
gaagtgcagctggtggaaagcggcggcggcctggtgcagccgggcggcagcctgcgcctgagctgcgcggcgagcggctttacctttagccgctattggatgagctgggtgcgccaggcgccgggcaaaggcctggaatgggtggcgaacattaaacaggatggcagcgaaaaatattatgtggatagcgtgaaaggccgctttaccattagccgcgataacgcgaaaaacagcctgtatctgcagatgaacagcctgcgcgcggaagataccgcggtgtattattgcgcgcgcgaaggcggctggtttggcgaactggcgtttgattattggggccagggcaccctggtgaccgtgagcagc

SEQ ID NO: 12 - DNA, 330 bp, synthetic construct
  source 1..330: mol_type = other DNA
cagagcgcgctgacccagccggcgagcgtgagcggcagcccgggccagagcattaccattagctgcaccggcaccagcagcgatgtgggcggctataactatgtgagctggtatcagcagcatccgggcaaagcgccgaaactgatgatttatgatgtgagcaaccgcccgagcggcgtgagcaaccgctttagcggcagcaaaagcggcaacaccgcgagcctgaccattagcggcctgcaggcggaagatgaagcggattattattgcagcagctataccagcagcagcacccgcgtgtttggcaccggcaccaaagtgaccgtgctg

SEQ ID NO: 13 - DNA, 360 bp, synthetic construct
  source 1..360: mol_type = other DNA
gaagtgcagctgctggaaagcggcggcggcctggtgcagccgggcggcagcctgcgcctgagctgcgcggcgagcggctttacctttagcagctatattatgatgtgggtgcgccaggcgccgggcaaaggcctggaatgggtgagcagcatttatccgagcggcggcattaccttttatgcggataccgtgaaaggccgctttaccattagccgcgataacagcaaaaacaccctgtatctgcagatgaacagcctgcgcgcggaagataccgcggtgtattattgcgcgcgcattaaactgggcaccgtgaccaccgtggattattggggccagggcaccctggtgaccgtgagcagc

SEQ ID NO: 14 - DNA, 787 bp, synthetic construct
  source 1..787: mol_type = other DNA
atcgataggtaccgagctcttacgcgtgctagccccgatgttttctgagttacttttgtatccccaccccccctcgaggaggaaaaactgtttcatacagaaggcgtacgccttctgtatgaaacagtttttcctccacgccttctgtatgaaacagtttttcctcctcgaggacattttgacacccccataatatttttccagaattaacagtataaattgcatctcttgttcaagagttccctatcactctctttaatcactactcacagtaacctcaactcctgcccaagcttggcattccggtactgttggtaaagccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 15 - protein, 21 aa, synthetic construct
IYIWAPLAGTCGVLLLSLVIT

SEQ ID NO: 16 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 17 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - DNA, 63 bp, synthetic construct
  source 1..63: mol_type = other DNA
atttatatttgggcgccgctggcgggcacctgcggcgtgctgctgctgagcctggtgattacc

SEQ ID NO: 19 - DNA, 765 bp, synthetic construct
  source 1..765: mol_type = other DNA
atgggcaacagctgctataacattgtggcgaccctgctgctggtgctgaactttgaacgcacccgcagcctgcaggatccgtgcagcaactgcccggcgggcaccttttgcgataacaaccgcaaccagatttgcagcccgtgcccgccgaacagctttagcagcgcgggcggccagcgcacctgcgatatttgccgccagtgcaaaggcgtgtttcgcacccgcaaagaatgcagcagcaccagcaacgcggaatgcgattgcaccccgggctttcattgcctgggcgcgggctgcagcatgtgcgaacaggattgcaaacagggccaggaactgaccaaaaaaggctgcaaagattgctgctttggcacctttaacgatcagaaacgcggcatttgccgcccgtggaccaactgcagcctggatggcaaaagcgtgctggtgaacggcaccaaagaacgcgatgtggtgtgcggcccgagcccggcggatctgagcccgggcgcgagcagcgtgaccccgccggcgccggcgcgcgaaccgggccatagcccgcagattattagcttttttctggcgctgaccagcaccgcgctgctgtttctgctgttttttctgaccctgcgctttagcgtggtgaaacgcggccgcaaaaaactgctgtatatttttaaacagccgtttatgcgcccggtgcagaccacccaggaagaagatggctgcagctgccgctttccggaagaagaagaaggcggctgcgaactg

SEQ ID NO: 20 - DNA, 492 bp, synthetic construct
  source 1..492: mol_type = other DNA
atgaaatggaaagcgctgtttaccgcggcgattctgcaggcgcagctgccgattaccgaagcgcagagctttggcctgctggatccgaaactgtgctatctgctggatggcattctgtttatttatggcgtgattctgaccgcgctgtttctgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcagcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 21 - protein, 96 aa, synthetic construct
QVQLQESGPGLVKPSETLSLTCTVSGGSISSYYWSWIRQPPGKGLEWIGRIYYSGSTYYNPSLKSRVTISVDTSKNQFSLKLSSVTAADTAVYYCA

SEQ ID NO: 22 - protein, 98 aa, synthetic construct
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCAR

SEQ ID NO: 23 - DNA, 288 bp, synthetic construct
  source 1..288: mol_type = other DNA
caggtgcagctgcaggaaagcggcccgggcctggtgaaaccgagcgaaaccctgagcctgacctgcaccgtgagcggcggcagcattagcagctattattggagctggattcgccagccgccgggcaaaggcctggaatggattggccgcatttattatagcggcagcacctattataacccgagcctgaaaagccgcgtgaccattagcgtggataccagcaaaaaccagtttagcctgaaactgagcagcgtgaccgcggcggataccgcggtgtattattgcgcg

SEQ ID NO: 24 - DNA, 294 bp, synthetic construct
  source 1..294: mol_type = other DNA
gaagtgcagctggtggaaagcggcggcggcctggtgcagccgggcggcagcctgcgcctgagctgcgcggcgagcggctttacctttagcagctatgaaatgaactgggtgcgccaggcgccgggcaaaggcctggaatgggtgagctatattagcagcagcggcagcaccatttattatgcggatagcgtgaaaggccgctttaccattagccgcgataacgcgaaaaacagcctgtatctgcagatgaacagcctgcgcgcggaagataccgcggtgtattattgcgcgcgc

(19) United States
(12) Patent Application Publication
(10) Pub. No.: US 2026/0345777 A1
(43) Pub. Date: Nov. 5, 2026

(54) CHIMERIC ANTIGEN RECEPTOR TARGETING PD-L1 AND T CELL THERAPEUTIC AGENT EXPRESSING SAME

(71) Applicant: AJOU UNIVERSITY INDUSTRY-ACADEMIC COOPERATION FOUNDATION, Suwon-si (KR)

(72) Inventors: Mi Ran KIM, Yongin-si (KR); Sang Rae LEE, Suwon-si (KR); Joo Hee KIM, Suwon-si (KR); Choon Ho PARK, Pohang-si (KR)

(73) Assignee: AJOU UNIVERSITY INDUSTRY-ACADEMIC COOPERATION FOUNDATION, Suwon-si (KR)

(21) Appl. No.: 19/403,874

(22) Filed: Nov. 30, 2025

Related U.S. Application Data
(63) Continuation of application No. PCT/KR2024/007365, filed on May 30, 2024.

(30) Foreign Application Priority Data
May 30, 2023 (KR) 10-2023-0069070
May 29, 2024 (KR) 10-2024-0069767

(51) Int. Cl. A61K 40/31; A61K 40/11; A61K 40/41; A61P 35/00; C07K 14/54; C07K 14/55; C07K 14/705; C07K 16/28; C12N 5/0783; C12N 15/86

(52) U.S. Cl. CPC A61K 40/31; A61K 40/11; A61K 40/41; A61P 35/00; C07K 14/54; C07K 14/5418; C07K 14/5434; C07K 14/5443; C07K 14/55; C07K 14/7051; C07K 14/70517; C07K 14/70578; C07K 16/2827; C12N 5/0636; C12N 15/86; C07K 2317/622; C07K 2319/02; C07K 2319/03; C12N 2510/00; C12N 2740/15043

(57) ABSTRACT

The present invention relates to a chimeric antigen receptor (CAR) binding to PD-L1 of tumor cells and a composition for treating cancer using T cells transformed with an anti-PD-L1 CAR and provides T cells expressing a chimeric antigen receptor binding specifically to PD-L1 overexpressed in various solid cancers. Moreover, in the present invention, T cells are provided with a means capable of selectively expressing a cytokine increasing anticancer efficacy together with a site encoding an anti-PD-L1 chimeric antigen receptor. In addition, the present invention provides a cell therapeutic composition for preventing or treating cancer, the composition comprising T cells expressing the anti-PD-L1 chimeric antigen receptor prepared according to the present invention.

### CROSS-REFERENCE TO RELATED APPLICATIONS

This application is a Continuation of PCT International Patent Application No. PCT/KR2024/007365 filed on May 30, 2024, under 35 U.S.C. § 371, which claims the benefit of Korean Patent Application Nos. 10-2023-0069070 filed on May 30, 2023 and 10-2024-0069767 filed on May 29, 2024, the entire contents of which are incorporated herein by reference.

### SEQUENCE LISTING

This application contains a Sequence Listing submitted via EFS-Web and hereby incorporated by reference in its entirety. The Sequence Listing is named \2280-627.xml, created Nov. 27, 2025, and 27,218 bytes in size.

### BACKGROUND

The present disclosure relates to a composition for treating a cancer using a chimeric antigen receptor (CAR) that binds to PD-L1 of a tumor cell and a T cell transformed with an anti-PD-L1 CAR.

A chimeric antigen receptor (CAR)-T cell has revolutionized the field of cell therapeutic agents by demonstrating effective, sustainable clinical therapeutic effects in hematological malignancies. Chimeric antigen receptors are engineered synthetic receptors to activate functions of T lymphocytes in an attempt to recognize and eliminate cancer cells expressing specific target antigens, and the binding of CARs to target antigens expressed on the cell surface shows active T cell activation and potent anticancer effects.

Based on the groundbreaking success of anti-CD19 CAR-T cells for B-cell blood cancers, the U.S. Food and Drug Administration (FDA) approved chimeric antigen receptor-based cell therapeutic agents in 2017. However, CAR-T cell therapeutic agents still bear many challenges to overcome, including CAR-T cell-induced toxicity that would threaten the life of patients, limited efficacy against solid tumors compared to hematological malignancies, resistance to B-cell malignancies, and limited persistence. Therefore, several different approaches have been recently attempted, including combination of CAR-T cell therapy with other anticancer therapies or use of innovative CAR engineering strategies to improve an anticancer efficacy, extension of duration of clinical efficacy, and attenuation of toxicity.

Programmed death-ligand 1 (PD-L1) is a 40 kDa integral membrane protein, and cancer cells in particular overexpress PD-L1 to evade attacks by tumor antigen-specific T cells. PD-L1, overexpressed in cancer cells, binds to PD-1, a receptor of PD-L1 that is expressed on tumor antigen-specifically activated T cells, and suppresses the activity of tumor antigen-specific T cells through PD-1/PD-L1 signaling. Therefore, antibody therapeutic agents (immune checkpoint inhibitors) capable of suppressing PD-1/PD-L1 signaling have been developed and currently actively utilized as therapeutic agents that are able to more effectively eliminate solid cancers. In addition, in order to increase the efficacy of CAR-T cells against solid tumors, a new concept of CAR-T cell therapeutic agents is being actively developed to overcome the tumor microenvironment of immunosuppressed solid tumors by expressing not only chimeric antigen receptors that bind to tumor cell antigens but also various interleukins or chemokines with anticancer effects.

Based on this, the inventors of the present disclosure took advantage of the fact that PD-L1 is overexpressed in various tumor cells to prepare CAR-T cells in which an interleukin gene that enhances an anticancer effect is additionally introduced into a chimeric antigen receptor using an antibody that binds to PD-L1. In this case, the interleukin gene is designed not to be constantly expressed but rather expressed at the time when T cells are activated so as to increase the killing ability of T cells while simultaneously activating other anti-tumor immune cells such as natural killer cells and macrophages, with side effects such as cytokine release syndrome minimized. CAR-T cells designed to have these enhanced functions were found to exhibit cytotoxicity specifically against tumor cells expressing PD-L1.

### PRIOR-ART DOCUMENT

### Patent Document

Korean Patent Application Publication No. 10-2019-0082241 (Published on Jul. 9, 2019)

### SUMMARY

An object of the present disclosure is to provide T cells expressing a chimeric antigen receptor that specifically binds to PD-L1 which is overexpressed in various solid cancers. Additionally, an object of the present disclosure is to provide T cells with a region encoding an anti-PD-L1 chimeric antigen receptor in addition to a means to selectively express cytokines that enhance an anticancer efficacy. In addition, an object of the present disclosure is to provide a cell therapy composition for preventing or treating a cancer, including a T cell expressing an anti-PD-L1 chimeric antigen receptor prepared according to the present disclosure.

The present disclosure provides a vector for preparing a T cell that expresses a chimeric antigen receptor, including an anti-PD-L1 scFv domain and an inducible cytokine domain.

In addition, the present disclosure provides a T cell that expresses a chimeric antigen receptor including an anti-PD-L1 scFv transfected with the vector while selectively expressing a cytokine depending on activation of T cells, and a cell therapy composition for preventing or treating a cancer using the same.

According to the present disclosure, an anti-PD-L1 chimeric antigen receptor including an anti-PD-L1 scFv domain is an antibody domain that specifically binds to PD-L1 that is specifically expressed in cancer cells, where T cells transformed with the anti-PD-L1 chimeric antigen receptor not only show improved specificity for tumor cells, but also have an inducible cytokine domain that is co-transformed and selectively expresses cytokines depending on activation of cells, such that it is possible to prepare T cells that express a chimeric antigen receptor with remarkably enhanced cytotoxicity.

### BRIEF DESCRIPTION OF THE DRAWINGS

FIG. 1 shows schematic diagrams illustrating DNA regions of each domain that expresses an anti-PD-L1 chimeric antigen receptor according to the present disclosure.

FIG. 2 shows a schematic diagram illustrating a DNA region of each domain additionally including an inducible cytokine domain in an anti-PD-L1 chimeric antigen receptor according to the present disclosure.

FIG. 3 shows a schematic diagram of a lentiviral vector for expressing an anti-PD-L1 chimeric antigen receptor construct according to the present disclosure.

FIG. 4 shows a schematic diagram illustrating expression of an anti-PD-L1 chimeric antigen receptor according to the present disclosure on the surface of a T cell.

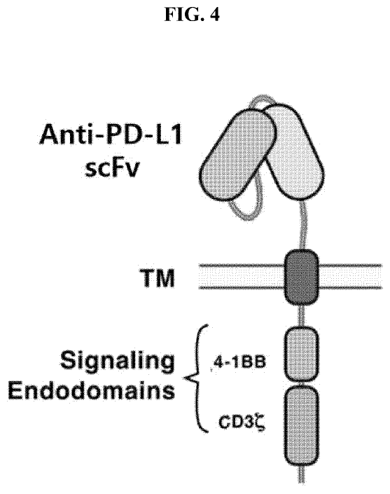

FIG. 5 shows a result of measuring a cytotoxic effect of T cells expressing an anti-PD-L1 chimeric antigen receptor according to the present disclosure on an MDA-MB-231 cell which is a PD-L1 expressing breast cancer cell line.

### DETAILED DESCRIPTION

The terms used in the present specification have been selected from general terms that are currently widely used as much as possible while considering the functions in the present disclosure, but these may vary depending on the intention of those skilled in the art or precedents or the emergence of new technologies. Additionally, in certain cases, there are terms arbitrarily selected by the applicant, and in such cases, meanings thereof will be set forth in detail in the detailed description of the disclosure. Therefore, the terms used herein should be defined based on the meaning of the terms and the overall content of the present disclosure, rather than simply the names of the terms.

Unless otherwise defined, all terms used herein, including technical or scientific terms, have the same meaning as commonly understood by those skilled in the art to which the present disclosure pertains. Terms defined in commonly used dictionaries should be interpreted as having a meaning consistent with the meaning in the context of the relevant art, but not be interpreted in an idealized or overly formal sense, unless explicitly defined in this application.

Hereinafter, the present disclosure will be described in more detail.

The present disclosure provides a vector for preparing a T cell that expresses a chimeric antigen receptor, including an anti-PD-L1 scFv domain and an inducible cytokine domain.

The chimeric antigen receptor includes an anti-PD-L1 scFv domain, a CD8 transmembrane (TM) domain, a 4-1BB domain, and a CD3 (zeta) domain, and is prepared as a vector including an antibody region binding to PD-L1, a cell membrane penetrating domain, and a domain for T cell transduction and activation.

The anti-PD-L1 scFv domain may include a light chain consisting of an amino acid sequence represented by SEQ ID NO: 1 and a heavy chain consisting of an amino acid sequence represented by SEQ ID NO: 2, or a light chain consisting of an amino acid sequence represented by SEQ ID NO: 3 and a heavy chain consisting of an amino acid sequence represented by SEQ ID NO: 4, or a light chain consisting of an amino acid sequence represented by SEQ ID NO: 5 and a heavy chain consisting of an amino acid sequence represented by SEQ ID NO: 6.

The anti-PD-L1 scFv domain may include sequentially a gene encoding a light chain of anti-PD-L1 scFv represented by SEQ ID NO: 8 and a gene encoding a heavy chain of anti-PD-L1 scFv represented by SEQ ID NO: 9, or sequentially include a gene encoding a light chain of anti-PD-L1 scFv represented by SEQ ID NO: 10 and a gene encoding a heavy chain of anti-PD-L1 scFv represented by SEQ ID NO: 11, or sequentially include a gene encoding a light chain of anti-PD-L1 scFv represented by SEQ ID NO: 12 and a gene encoding a heavy chain of anti-PD-L1 scFv represented by SEQ ID NO: 13.

The vector further includes, in sequence following the anti-PD-L1 scFv domain, a CD8 transmembrane (TM) domain, a 4-1BB domain, and a CD3ζ (zeta) domain. The CD8 transmembrane (TM) domain consists of a base sequence represented by SEQ ID NO: 18, the 4-1BB domain consists of a base sequence represented by SEQ ID NO: 19, and the CD3ζ (zeta) domain consists of a base sequence represented by SEQ ID NO: 20.

The inducible cytokine domain sequentially includes a nuclear factor of activated T cell (NFAT) binding motif and a gene encoding a cytokine. The cytokine may be any one selected from IL-2, IL-12, IL-7, IL-15, or IL-18.

Specifically, the inducible cytokine domain may consist of a base sequence represented by SEQ ID NO: 14 that sequentially includes a nuclear factor of activated T cell (NFAT) binding motif and a nucleotide encoding IL-2 so as to selectively express IL-2.

In addition, the present disclosure provides a T cell that expresses a chimeric antigen receptor including an anti-PD-L1 scFv transfected with the vector while selectively expressing a cytokine depending on activation of T cells.

The T cell is a T cell of CD4⁺/CD8⁺ type isolated from mononuclear cells and stimulated with anti-CD3 and anti-hCD28.

In addition, the present disclosure provides a pharmaceutical composition for preventing or treating a cancer, including a transformed T cell which expresses a chimeric antigen receptor including an anti-PD-L1 scFv prepared according to the present disclosure on a surface and includes an inducible cytokine domain. In addition, the present disclosure provides a method for treating a cancer in a subject in need thereof, comprising administering to the subject a therapeutically effective amount of the pharmaceutical composition.

The cancer may be any one or more diseases selected from leukemia, acute lymphoblastic leukemia, acute myeloblastic leukemia, chronic leukemia, chronic myeloblastic (granulocytic) leukemia, chronic lymphocytic leukemia, polycythemia vera, lymphoma, Hodgkin's disease, non-Hodgkin's disease, multiple myeloma, Waldenstrom's macroglobulinemia, heavy chain disease, fibrosarcoma, myxosarcoma, liposarcoma, chondrosarcoma, osteosarcoma, chordoma, angiosarcoma, endothelial sarcoma, lymphangiosarcoma, lymphangio endothelial sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, cholangiocarcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The pharmaceutical composition may be prepared in unit dose form by formulating it using a pharmaceutically acceptable carrier or prepared by placing it in a multi-dose container.

The pharmaceutically acceptable carriers are those commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additive included in the pharmaceutical composition is not particularly limited and may be appropriately adjusted within the content range used in conventional formulations.

In the present disclosure, the vector is a tool that enables stable transfection of cells, and the vector allows incorporation of transgene(s) into a cell genome. Preferably, such vectors have a positive selection marker, and a suitable positive selection marker includes any gene that allows cells to grow under conditions for killing cells that do not express the gene. Non-limiting examples may include antibiotic resistance. Additionally, the vector is a plasmid vector, and more specifically, the vector is a viral vector and may be utilized in replace of any suitable vector by those skilled in the art, and preferably, the vector may be a lentivirus vector, a retrovirus vector, a DNA vector, a plasmid, an RNA vector, an adenovirus vector, or an adenovirus-associated vector, but is not limited thereto.

As used herein, the term “protein” is used interchangeably with “peptide” and “polypeptide” and refers to a compound having amino acid residues covalently linked by peptide bonds. A protein or peptide must include at least two amino acids, and there is no limit to the maximum number of amino acids that may be included in the sequence of a protein or peptide. A polypeptide includes any peptide or protein having two or more amino acids linked one another by peptide bonds. The terms as used herein refer to both chains, where, for example, the shorter chains are also commonly referred to as peptides, oligopeptides, and oligomers in the art, whereas the longer chains are commonly referred to as proteins in the art, and many types have belonged thereto. The term “polypeptide” as used herein includes, for example, biologically active fragments, substantially homologous polypeptides, oligopeptides, homodimers, heterodimers, variants of polypeptides, modified polypeptides, derivatives, analogs, and fusion proteins. The polypeptide includes a natural peptide, a recombinant peptide, a synthetic peptide, or a combination thereof.

In the present disclosure, the nucleotide is commonly used in the same meaning as polynucleotide, and includes DNA and RNA. The polynucleotide is a polymer of nucleotides as a nucleic acid, and the polynucleotide may be hydrolyzed into monomeric nucleotides. The polynucleotide includes, but is not limited to, all nucleic acid sequences obtained by recombination means, that is, recombinant libraries using conventional cloning techniques and polymerase chain reaction (PCR) or any means available in the art, including cloning of nucleic acid sequences from cellular genomes and synthetic means.

### MODES FOR CARRYING OUT THE INVENTION

Hereinafter, in order to help understand the present disclosure, experimental examples and examples will be described in detail. However, the following experimental examples and examples are only illustrative of the contents of the present disclosure, and the scope of the present disclosure is not limited to the following experimental examples and examples. The experimental examples and examples of the present disclosure are provided to more completely describe the present disclosure to those with ordinary skill in the art.

### Example 1. Preparation of Anti-PD-L1 Chimeric Antigen Receptor

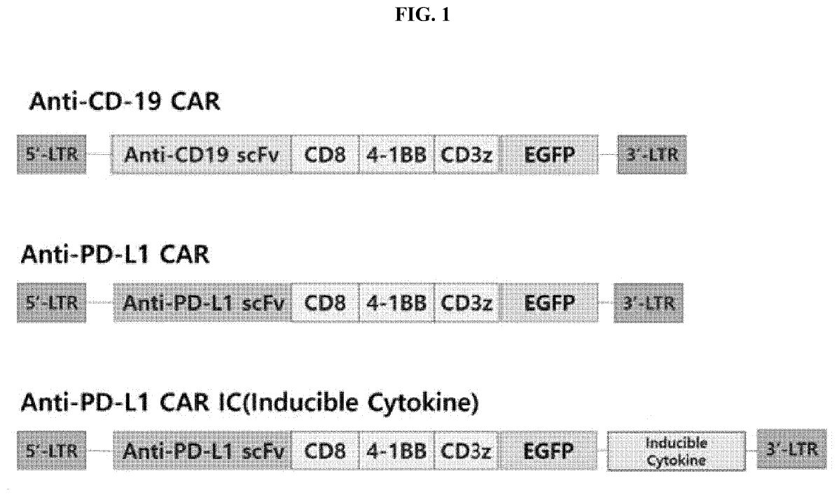

For the anti-PD-L1 chimeric antigen receptor (hereinafter referred to as CAR) according to the present disclosure, a genetic construct encoding the anti-PD-L1 CAR was prepared in reference to the anti-CD-19 CAR structure based on a genetic database as shown in FIG. 1. In this Example, as one aspect of the CAR, design was made to sequentially include a single chain variable fragment (scFv) domain that binds to PD-L1, a CD8 transmembrane (TM) domain, a 4-1BB domain, and a CD3ζ (zeta) domain. The scFv domain was designed to be exposed outside the cell and prepared in three types to have the configurations shown in Table 1 below. Specifically, the anti-PD-L1 CAR according to the present disclosure may be of type A consisting of a light chain consisting of an amino acid sequence represented by SEQ ID NO: 1 and a heavy chain consisting of an amino acid sequence represented by SEQ ID NO: 2 as an anti-PD-L1 scFv domain, type B consisting of a light chain consisting of an amino acid sequence represented by SEQ ID NO: 3 and a heavy chain consisting of an amino acid sequence represented by SEQ ID NO: 4, or type C consisting of a light chain consisting of an amino acid sequence represented by SEQ ID NO: 5 and a heavy chain consisting of an amino acid sequence represented by SEQ ID NO: 6.

TABLE 1

| | | | SEQ ID |
|---|---|---|---|
| Type | | Amino acid sequence | NO: |
| A | Light chain | DIQMTQSPSSLSASVGDRVTITCRASQDVSTAVA | 1 |
| | | WYQQKPGKAPKLLIYSASFLYSGVPSRFSGSGSGT | |
| | | DFTLTISSLQPEDFATYYCQQYLYHPATFGQGTKV | |
| | | EIK | |
| | Heavy chain | EVQLVESGGGLVQPGGSLRLSCAASGFTFSDSWIH | 2 |
| | | WVRQAPGKGLEWVAWISPYGGSTYYADSVKGRF | |
| | | TISADTSKNTAYLQMNSLRAEDTAVYYCARRHW | |
| | | PGGFDYWGQGTLVTVSS | |
| B | Light chain | EIVLTQSPGTLSLSPGERATLSCRASQRVSSSYLA | 3 |
| | | WYQQKPGQAPRLLIYDASSRATGIPDRFSGSGSGT | |
| | | DFTLTISRLEPEDFAVYYCQQYGSLPWTFGQGTK | |
| | | VEIK | |
| | Heavy chain | EVQLVESGGGLVQPGGSLRLSCAASGFTFSRYWM | 4 |
| | | SWVRQAPGKGLEWVANIKQDGSEKYYVDSVKGR | |
| | | FTISRDNAKNSLYLQMNSLRAEDTAVYYCAREGG | |
| | | WFGELAFDYWGQGTLVTVSS | |
| C | Light chain | QSALTQPASVSGSPGQSITISCTGTSSDVGGYNYVS | 5 |
| | | WYQQHPGKAPKLMIYDVSNRPSGVSNRFSGSKSG | |
| | | NTASLTISGLQAEDEADYYCSSYTSSSTRVFGTGT | |
| | | KVTVL | |
| | Heavy chain | EVQLLESGGGLVQPGGSLRLSCAASGFTFSSYIMM | 6 |
| | | WVRQAPGKGLEWVSSIYPSGGITFYADTVKGRFTI | |
| | | SRDNSKNTLYLQMNSLRAEDTAVYYCARIKLGTV | |
| | | TTVDYWGQGTLVTVSS | |

The anti-PD-L1 CAR according to the present disclosure includes a CD8 transmembrane (TM) domain as a cell membrane-penetrating region. The CD8 transmembrane (TM) domain includes an amino acid sequence represented by SEQ ID NO: 15. The anti-PD-L1 CAR according to the present disclosure includes a 4-1BB domain consisting of an amino acid sequence represented by SEQ ID NO: 16 and a CD35 (zeta) domain consisting of an amino acid sequence represented by SEQ ID NO: 17, wherein the 4-1BB domain and the CD3ζ (zeta) domain are designed to be expressed inside a cell.

As shown in FIG. 1, the anti-PD-L1 CAR gene construct according to the present disclosure was prepared to sequentially include a gene encoding a light chain of anti-PD-L1 scFv, a linker sequence, a gene encoding a heavy chain of anti-PD-L1 scFv, a CD8 transmembrane (TM) domain, a 4-1BB domain, and a CD3ζ (zeta) domain.

The CAR gene construct type A includes a gene encoding a light chain of anti-PD-L1 scFv represented by SEQ ID NO: 8, and a gene encoding a heavy chain of anti-PD-L1 scFv represented by SEQ ID NO: 9.

The CAR gene construct type B includes a gene encoding a light chain of anti-PD-L1 scFv represented by SEQ ID NO: 10, and a gene encoding a heavy chain of anti-PD-L1 scFv represented by SEQ ID NO: 11.

The CAR gene construct type C includes a gene encoding a light chain of anti-PD-L1 scFv represented by SEQ ID NO: 12, and a gene encoding a heavy chain of anti-PD-L1 scFv represented by SEQ ID NO: 13.

The CD8 transmembrane (TM) domain consists of a base sequence represented by SEQ ID NO: 18. The 4-1BB domain consists of a base sequence represented by SEQ ID NO: 19. The CD3ζ (zeta) domain consists of a base sequence represented by SEQ ID NO: 20.

Additionally, the anti-PD-L1 CAR gene construct according to the present disclosure may further include an inducible cytokine domain. The inducible cytokine domain is not constitutively expressed but is designed to be expressed depending on the activity of T cells infected with the chimeric antigen receptor according to the present disclosure and prevent toxicity and inflammation caused by excessive cytokines.

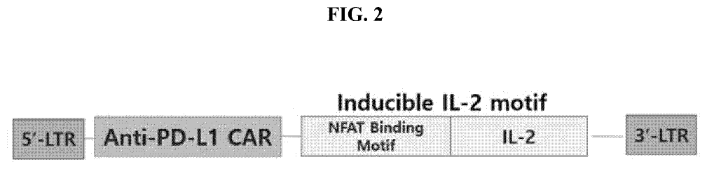

In one embodiment of the present disclosure, as shown in FIG. 2, the 3′ end of the anti-PD-L1 CAR configuration includes an inducible IL-2 domain which sequentially includes a nuclear factor of activated T cell (NFAT) binding motif and a nucleotide encoding IL-2. The gene encoding the inducible IL-2 domain consists of a base sequence represented by SEQ ID NO: 14. The gene encoding the inducible IL-2 domain may selectively induce expression of interleukin-2 (IL-2) by including a nuclear factor of activated T cell (NFAT) binding motif, and the IL-2 expressed by the inducible IL-2 domain consists of an amino acid sequence represented by SEQ ID NO: 7.

### Example 2. Preparation of Vector for Expressing Anti-PD-L1 CAR and Lentivirus

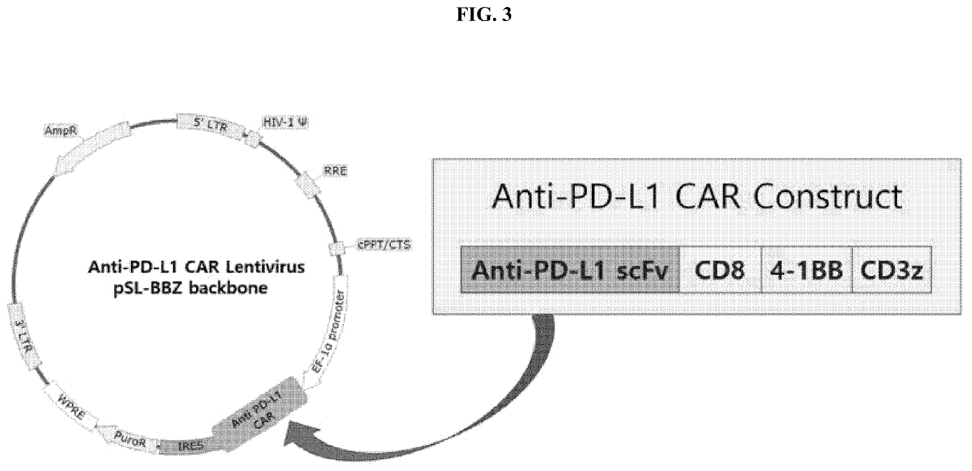

A vector for expressing the anti-PD-L1 CAR according to the present disclosure was prepared, and a lentivirus was used as a means of delivering the same. As shown in FIG. 3, the third-generation lentiviral vector pSL-BBZ was used as the basic backbone. The anti-PD-L1 CAR construct of the present disclosure was inserted into the vector.

In the anti-PD-L1 CAR construct, a gene encoding the light chain of anti-PD-L1 scFv and a gene encoding the heavy chain of anti-PD-L1 scFv are located between the Bam H1 site and the Xho I site. Additionally, to enable experimental detection of expression, a sequence of enhanced green fluorescent protein (EGFP) was designed to be expressed at the end of the CAR construct.

The synthesized gene was subcloned into a lentiviral plasmid vector via the polymerase chain reaction. Finally, the accuracy of the gene sequence was identified by base sequencing using specific primers of the cloned plasmid. In addition, the nucleotide sequences and regulatory regions of cytokine genes such as IL-2, IL-12, IL-7, IL-15, or IL-18 were synthesized by identification from a genetic database, and subcloned into a lentiviral plasmid vector by the same method as above to ultimately fabricate as an anti-PD-L1 CAR construct.

In this Example, an anti-PD-L1 CAR construct was fabricated to sequentially include an inducible IL-2 domain that sequentially includes a nuclear factor of activated T cell (NFAT) binding motif and a nucleotide encoding IL-2 in the CAR gene construct type C.

3 μg of lentiviral vector designed to include the anti-PD-L1 CAR construct was transfected into HEK293T cells (5×10⁶ cells) using Lipofectamine (Thermo Scientific #R0531). After culturing for 48 hours, the supernatant was collected and purified through a 0.45 μm filter to remove only the virus particles. The purified virus particles were concentrated by ultracentrifugation at 28,000 rpm for 2 hours.

### Example 3. Isolation and Activation of Human T Cells

To prepare T cells expressing the anti-PD-L1 CAR according to the present disclosure, mononuclear cells were isolated from human peripheral blood. CD8⁺ T cells were isolated from peripheral blood mononuclear cells by magnetic cell sorting (MACS), and prepared as isolated CD4⁻/CD8⁺/CD3⁺ T cells by stimulating with anti-hCD3 and anti-hCD28-coated immunobeads at a bead-to-cell ratio of 1:1 for 24 to 36 hours.

### Example 4. Preparation of T Cells Expressing Anti-PD-L1 CAR

In Example 3 above, activated T cells were cultured with concentrated anti-CAR lentiviral vectors in a 48-well flat plate coated with Novo Nectin (Novoprotein) at 32 °C. Six to seven days after transduction, the immunobeads were removed, and the T cells were adjusted to a cell density of 0.5×10⁶/ml to 2×10⁶/mL every three days.

### Example 5. Cytotoxicity of Anti-PD-L1 CAR T Cell Against PD-L1⁺ Cancer Cell Line

In order to identify the killing effect of T cells expressing the anti-PD-L1 CAR that is prepared according to the present disclosure on cancer cells, evaluation was conducted on the cancer killing effect on MDA-MB-231 cells, a breast cancer cell line. For comparison, T cells expressing anti-CD-19 CAR were used as a control. As shown in FIG. 1, an anti-CD-19 CAR configuration was used for the anti-CD-19 CAR, and the CAR-expressing T cells were prepared using lentivirus in the same manner as the anti-PD-L1 CAR. MDA-MB-231 cells were placed in a 96-well plate at 2×10⁴ cell/mL and cultured in Dulbecco's Modified Eagle's Medium (DMEM) containing FBS for 24 hours. T cells transformed with each CAR were added to the wells at various ratios and cultured at 37 °C for 4 or 24 hours. The medium was removed from the wells where the culture was completed, and 50 μl of 0.2% MTT (4,5-dimethylthiazol-2-yl)-2,5-diphenyltetrazolium) was added per well, followed by culture in an incubator at 37 °C for additional 2 hours. After removing the supernatant, 100 μl/well of dimethyl sulfoxide (DMSO) was added and suspended for 5 minutes to dissolve the resulting formazan, and the absorbance was measured at 570 nm using a microplate reader to determine the death rate of cancer cells.

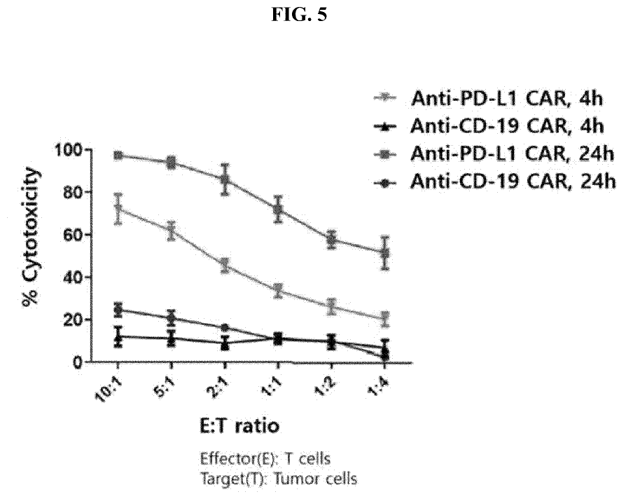

As shown in FIG. 5, when T cells transformed with each CAR were reacted with cancer cells, it was found that cytotoxicity did not change significantly at 4 hours, despite changes in the ratio between effector (E) T cells and target (T) cancer cells (E:T ratio). In the case of a 24-hour reaction, the death rate of cancer cells reacting with anti-CD-19 CAR T cells was found to decrease rapidly when the E:T ratio changed from 10:1 to 1:4. On the other hand, the death rate of cancer cells reacting with anti-PD-L1 CAR T cells was found to be significantly superior even when the E:T ratio changed from 10:1 to 1:4.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

The numerical range includes the numerical values defined in the above range. All maximum numerical limits given throughout the specification include all lower numerical limits as if the lower numerical limits were expressly written. All minimum numerical limits given throughout this specification include all higher numerical limits as if the higher numerical limits were expressly written. All numerical limits given throughout this specification will include all the better numerical range within the broader numerical range, as if the narrower numerical limits were explicitly written.

What is claimed is:

1. A vector for preparing a T cell expressing a chimeric antigen receptor (CAR), comprising an anti-PD-L1 scFv domain and an inducible cytokine domain.

2. The vector of claim 1, wherein the chimeric antigen receptor comprises an anti-PD-L1 scFv domain, a CD8 transmembrane (TM) domain, a 4-1BB domain, and a CD3ζ (zeta) domain.

3. The vector of claim 2, wherein the anti-PD-L1 scFv domain comprises:
a light chain consisting of an amino acid sequence represented by SEQ ID NO: 1 and a heavy chain consisting of an amino acid sequence represented by SEQ ID NO: 2;
a light chain consisting of an amino acid sequence represented by SEQ ID NO: 3 and a heavy chain consisting of an amino acid sequence represented by SEQ ID NO: 4; or
a light chain consisting of an amino acid sequence represented by SEQ ID NO: 5 and a heavy chain consisting of an amino acid sequence represented by SEQ ID NO: 6.

4. The vector of claim 1, wherein the anti-PD-L1 scFv domain comprises, in sequential order:
a gene encoding a light chain of anti-PD-L1 scFv represented by SEQ ID NO: 8 and a gene encoding a heavy chain of anti-PD-L1 scFv represented by SEQ ID NO: 9;
a gene encoding a light chain of anti-PD-L1 scFv represented by SEQ ID NO: 10 and a gene encoding a heavy chain of anti-PD-L1 scFv represented by SEQ ID NO: 11; or
a gene encoding a light chain of anti-PD-L1 scFv represented by SEQ ID NO: 12 and a gene encoding a heavy chain of anti-PD-L1 scFv represented by SEQ ID NO: 13.

5. The vector of claim 1, wherein the vector further comprises, in sequence following the anti-PD-L1 scFv domain, a CD8 transmembrane (TM) domain, a 4-1BB domain, and a CD3ζ (zeta) domain.

6. The vector of claim 5, wherein the CD8 transmembrane (TM) domain consists of a base sequence represented by SEQ ID NO: 18,
the 4-1BB domain consists of a base sequence represented by SEQ ID NO: 19, and
the CD3ζ (zeta) domain consists of a base sequence represented by SEQ ID NO: 20.

7. The vector of claim 1, wherein the inducible cytokine domain sequentially comprises a nuclear factor of activated T cell (NFAT) binding motif and a gene encoding a cytokine.

8. The vector of claim 7, wherein the cytokine is any one selected from IL-2, IL-12, IL-7, IL-15, or IL-18.

9. The vector of claim 1, wherein the inducible cytokine domain consists of a base sequence represented by SEQ ID NO: 14.

10. The vector of claim 1, wherein the vector is a lentiviral vector.

11. A T cell transfected with the vector of claim 1.

12. The T cell of claim 11, wherein the T cell is a T cell of CD4⁺/CD8⁺ type isolated from mononuclear cells and stimulated with anti-CD3 and anti-hCD28.

13. A method for treating a cancer in a subject in need thereof, comprising administering to the subject a therapeutically effective amount of a pharmaceutical composition comprising the T cell according to claim 11.
